(19) United States
(12) Patent Application Publication
(10) Pub. No.: US 2023/0190670 A1
(43) Pub. Date: Jun. 22, 2023

(54) COMPOSITIONS COMPRISING NOVEL CANNABINOID AND TERPENE PROFILES

(71) Applicant: Natural Extraction Systems, LLC, Boulder, CO (US)

(72) Inventor: C. Russell Thomas, Boulder, CO (US)

(73) Assignee: Natural Extraction Systems, LLC, Boulder, CO (US)

(21) Appl. No.: 17/784,059

(22) PCT Filed: Dec. 1, 2020
(86) PCT No.: PCT/US2020/062744

§ 371 Date: Jun. 9, 2022

Related U.S. Application Data
(60) Provisional application No. 62/942,445, filed on Dec. 2, 2019.

(51) Int. Cl. A61K 31/05; A61K 31/015; A61K 31/336; A61K 31/352; A61K 31/047

(52) U.S. Cl. CPC A61K 31/05; A61K 31/015; A61K 31/047; A61K 31/336; A61K 31/352

(57) ABSTRACT

This patent document discloses compositions comprising novel combinations of cannabinoids, terpenes, and thermal degradation products that can be obtained, for example, by the vapor-phase decarboxylation of a cannabis extract.

### CROSS-REFERENCE TO RELATED APPLICATIONS

This patent application claims priority to U.S. Provisional Pat Application No. 62/942,445, filed Dec. 2, 2019, which is incorporated by reference in its entirety.

### BACKGROUND

Cannabis flowers biosynthetically produce cannabigerolic acid, which is a carboxylic acid. The flowers can then cyclize the cannabigerolic acid into cannabidiolic acid, tetrahydrocannabinolic acid, and cannabichromenic acid at varying ratios depending on the phenotype of the cannabis strain. Cannabinoid carboxylic acids are generally decarboxylated to produce cannabigerol, cannabidiol, tetrahydrocannabinol, and cannabichromene by heating at a temperature greater than 100° C. for a period of time ranging from 30 minutes for boutique production to more than 6 hours for large-scale commercial production. Heating results in thermal degradation including the oxidation of cannabinoids into cannabinol and a significant loss of cannabigerolic acid (see, for example, Wang et al., CANNABIS AND CANNABINOID RESEARCH, 2016, 1(1):262, which suggests that greater than 50 percent of cannabigerolic acid is lost during decarboxylation performed for 60 minutes at temperatures ranging from 80 to 145° C.).

Cannabis also biosynthetically produces a variety of terpenes. The precise terpene content of a decarboxylated cannabis extract depends upon (1) the strain of cannabis; (2) the cannabis extraction method; (3) the cannabinoid decarboxylation method; and (4) purification methods such as solvent removal, distillation, carbon-filtering, and chromatography. Many terpenes are thermally degraded during decarboxylation.

Cannabinoid compositions that lack significant thermal degradation products are desirable.

### SUMMARY

This patent document discloses compositions comprising novel combinations of cannabinoids, terpenes, and thermal degradation products that can be obtained, for example, by the vapor-phase decarboxylation of a cannabis extract.

### DETAILED DESCRIPTION

Methods were previously developed to decarboxylate cannabinoid carboxylic acids in the vapor phase in approximately two seconds, which is a substantial improvement over conventional liquid-phase decarboxylation (see, for example, U.S. Pat No. 10,669,248). The improved methods (1) minimize the thermal degradation of an extract; (2) recover high concentrations of terpenes; and (3) reduce the need for post-decarboxylation purification. Products made from industrial hemp extract that is decarboxylated in the vapor phase display significantly increased pharmacological efficacy per milligram of cannabidiol relative to other commercial products across a wide range of medical conditions including anxiety, pain, autism spectrum disorders, and Down syndrome.

This patent document discloses novel features of the chemical fingerprint of cannabis extracts that underwent vapor-phase decarboxylation, including: (1) the relative abundance of cannabigerol and cannabigerovarin, which are recovered at higher yields using vapor-phase decarboxylation relative to liquid-phase decarboxylation; (2) the relative absence of cannabinol and cannabivarin, which are oxidation products of tetrahydrocannabinol and tetrahydrocannabivarin, respectively, that are produced during decarboxylation; (3) the relative abundance of terpenes, which depend upon numerous variables that include limited exposure to elevated temperatures during decarboxylation; and (4) the relative amounts of terpene degradation products, which vary depending upon exposure to elevated temperatures during decarboxylation and numerous other factors. This patent document also discloses additional characteristics that help differentiate cannabinoid extracts that are decarboxylated in the vapor phase from those that are decarboxylated in the liquid phase.

While each of the disclosed and claimed compositions may be obtained by the vapor-phase decarboxylation of a cannabis extract, the compositions may also be prepared by combining purified molecules at the ratios disclosed in this patent document.

Table 1 sets forth common names and corresponding chemical names featured in this patent document, and molecular structures for each species along with predominant stereochemistries are set forth in PCT Patent Application Publication No. WO 2020/123383 A1, which is incorporated by reference in its entirety.

TABLE 1

Common Names and Corresponding Chemical Names of Various Molecules

| | |
|---|---|
| Common Name | Chemical Name |
| Cannabidiol | 2-(6-isopropenyl-3-methylcyclohex-2-enyl)-5-pentylbenzene-1,3-diol |
| Cannabidivarin | 2-(6-isopropenyl-3-methylcyclohex-2-enyl)-5-propylbenzene-1,3-diol |
| Tetrahydrocannabinol | 6,6,9-trimethyl-3-pentyl-6a,7,8,10a-tetrahydro-6H-benzo[c]chromen-1-ol |
| Tetrahydrocannabivarin | 6, 6,9-trimethyl-3-propyl-6a,7,8,10a-tetrahydro-6H-benzo[c]chromen-1-ol |
| Cannabigerol | 2-(3,7-dimethylocta-2,6-dieneyl)-5-pentylbenzene-1,3-diol |
| Cannabigerovarin | 2-(3,7-dimethylocta-2,6-dieneyl)-5-propylbenzene-1,3-diol |
| Cannabichromene | 2-methyl-2-(4-methylpent-3-enyl)-7-pentyl-2H-1-benzopyran-5-ol |
| Cannabichromevarin | 2-methyl-2-(4-methylpent-3-enyl)-7-propyl-2H-1-benzopyran-5-ol |
| Cannabinol | 6,6,9-trimethyl-3-pentyl-6H-benzo[c]chromen-1-ol |
| Cannabivarin | 6,6,9-trimethyl-3-propyl-6H-benzo[c]chromen-1-ol |
| Beta-caryophyllene | 4,11,11-trimethyl-8-methylidenebicyclo[7.2.0]undec-4-ene |
| Caryophyllene oxide | 4,12,12-trimethyl-9-methylidene-5-oxatricyclo[8.2.0.0^{4,6}]dodecane |
| Humulene | 2,6,6,9-tetramethyl-1,4,8-cycloundecatriene |
| Humulene epoxide I | 1,5,9,9-tetramethyl-12-oxabicyclo[9.1.0]dodeca-4,7-diene |
| Humulene epoxide II | 1,5,5,8-tetramethyl-12-oxabicyclo[9.1.0]dodeca-3,7-diene |
| Humulene epoxide III | 3,7,10, 10-tetramethyl-12-oxabicyclo[9.1.0]dodeca-3,7-diene |
| Bicyclohumuladiol | 1, 5, 8, 8-tetramethylbicyclo[8.1.0]undec-5-ene-2, 9-diol |
| Tricyclohumuladiol | 4,8,11,11-tetramethyltricyclo[7.2.0.0^{2,4}]undecane-5,8-diol |
| Tricyclohumuladiol II | 4,8,11,11-tetramethyltricyclo[6.3.0.0^{2,4}]undecane-5,9-diol |
| Humulenol II | 1,4,4-trimethyl-8-methylidene-1,5-cycloundecadien-9-ol |

Various aspects of this patent document relate to a composition, comprising cannabigerovarin, beta-caryophyllene, caryophyllene oxide, humulene, and humulene epoxide II, wherein: the composition comprises the cannabigerovarin and the beta-caryophyllene at a ratio of at least 1:5 and no greater than 100:1 by mass; the composition comprises the cannabigerovarin and the caryophyllene oxide at a ratio of at least 1:1 and no greater than 500:1 by mass; the composition comprises the cannabigerovarin and the humulene at a ratio of at least 1:5 and no greater than 100:1 by mass; and the composition comprises the cannabigerovarin and the humulene epoxide II at a ratio of at least 1:1 and no greater than 500:1 by mass.

Various aspects of this patent document relate to a composition, comprising cannabidivarin, beta-caryophyllene, caryophyllene oxide, humulene, and humulene epoxide II, wherein: the composition comprises the cannabidivarin and the beta-caryophyllene at a ratio of at least 1:5 and no greater than 100:1 by mass; the composition comprises the cannabidivarin and the caryophyllene oxide at a ratio of at least 1:1 and no greater than 500:1 by mass; the composition comprises the cannabidivarin and the humulene at a ratio of at least 1:5 and no greater than 100:1 by mass; and the composition comprises the cannabidivarin and the humulene epoxide II at a ratio of at least 1:1 and no greater than 500:1 by mass.

Various aspects of this patent document relate to a composition, comprising tetrahydrocannabivarin, beta-caryophyllene, caryophyllene oxide, humulene, and humulene epoxide II, wherein: the composition comprises the tetrahydrocannabivarin and the beta-caryophyllene at a ratio of at least 1:5 and no greater than 100:1 by mass; the composition comprises the tetrahydrocannabivarin and the caryophyllene oxide at a ratio of at least 1:1 and no greater than 500:1 by mass; the composition comprises the tetrahydrocannabivarin and the humulene at a ratio of at least 1:5 and no greater than 100:1 by mass; and the composition comprises the tetrahydrocannabivarin and the humulene epoxide II at a ratio of at least 1:1 and no greater than 500:1 by mass.

Various aspects of this patent document relate to a composition, comprising cannabigerol, beta-caryophyllene, caryophyllene oxide, humulene, and humulene epoxide II, wherein: the composition comprises the cannabigerol and the beta-caryophyllene at a ratio of at least 1:5 and no greater than 100:1 by mass; the composition comprises the cannabigerol and the caryophyllene oxide at a ratio of at least 1:1 and no greater than 500:1 by mass; the composition comprises the cannabigerol and the humulene at a ratio of at least 1:5 and no greater than 100:1 by mass; and the composition comprises the cannabigerol and the humulene epoxide II at a ratio of at least 1:1 and no greater than 500:1 by mass.

Various aspects of this patent document relate to a composition, comprising cannabidiol, beta-caryophyllene, caryophyllene oxide, humulene, and humulene epoxide II, wherein: the composition comprises the cannabidiol and the beta-caryophyllene at a ratio of at least 1:1 and no greater than 500:1 by mass; the composition comprises the cannabidiol and the caryophyllene oxide at a ratio of at least 10:1 and no greater than 5,000:1 by mass; the composition comprises the cannabidiol and the humulene at a ratio of at least 1:1 and no greater than 500:1 by mass; and the composition comprises the cannabidiol and the humulene epoxide II at a ratio of at least 10:1 and no greater than 5,000:1 by mass.

Various aspects of this patent document relate to a composition, comprising tetrahydrocannabinol, beta-caryophyllene, caryophyllene oxide, humulene, and humulene epoxide II, wherein: the composition comprises the tetrahydrocannabinol and the beta-caryophyllene at a ratio of at least 1:1 and no greater than 500:1 by mass; the composition comprises the tetrahydrocannabinol and the caryophyllene oxide at a ratio of at least 10:1 and no greater than 5,000:1 by mass; the composition comprises the tetrahydrocannabinol and the humulene at a ratio of at least 1:1 and no greater than 500:1 by mass; and the composition comprises the tetrahydrocannabinol and the humulene epoxide II at a ratio of at least 10:1 and no greater than 5,000:1 by mass.

In some embodiments, the composition comprises one, two, three or each of cannabigerol, cannabidiol, tetrahydrocannabinol, and cannabichromene. In some specific embodiments, the composition comprises cannabigerol, cannabidiol, tetrahydrocannabinol, and cannabichromene.

In some embodiments, the composition comprises cannabigerol and tetrahydrocannabinol at a ratio of greater than 1:1,000 and no greater than 1,000:1 by mass. In some specific embodiments, the composition comprises cannabigerol and tetrahydrocannabinol at a ratio of greater than 1:1 and no greater than 1,000:1 by mass. In some very specific embodiments, the composition comprises cannabigerol and tetrahydrocannabinol at a ratio of greater than 10:1 and no greater than 1,000:1 by mass.

In some embodiments, the composition comprises cannabidiol and cannabinol at a ratio of at least 1:1 and no greater than 10,000:1 by mass. In some specific embodiments, the composition comprises cannabidiol and cannabinol at a ratio of at least 100:1 and no greater than 10,000:1 by mass. In some very specific embodiments, the composition comprises cannabidiol and cannabinol at a ratio of at least 100:1 and no greater than 1,000:1 by mass.

In some embodiments, the composition comprises one, two, three, or each of cannabigerovarin, cannabidivarin, tetrahydrocannabivarin, and cannabichromevarin. In some specific embodiments, the composition comprises cannabigerovarin, cannabidivarin, tetrahydrocannabivarin, and cannabichromevarin.

In some embodiments, the composition comprises cannabigerovarin and tetrahydrocannabivarin at a ratio of greater than 1:1,000 and no greater than 1,000:1 by mass. In some specific embodiments, the composition comprises cannabigerovarin and tetrahydrocannabivarin at a ratio of greater than 1:1 and no greater than 1,000:1 by mass. In some very specific embodiments, the composition comprises cannabigerovarin and tetrahydrocannabivarin at a ratio of greater than 10:1 and no greater than 1,000:1 by mass.

In some embodiments, the composition comprises cannabidivarin and cannabivarin at a ratio of at least 1:1 and no greater than 10,000:1 by mass. In some specific embodiments, the composition comprises cannabidivarin and cannabivarin at a ratio of at least 100:1 and no greater than 10,000:1 by mass. In some very specific embodiments, the composition comprises cannabidivarin and cannabivarin at a ratio of at least 100:1 and no greater than 1,000:1 by mass.

In some embodiments, the composition comprises beta-caryophyllene and caryophyllene oxide at a ratio of at least 1:1 and no greater than 1,000:1 by mass. In some specific embodiments, the composition comprises beta-caryophyllene and caryophyllene oxide at a ratio of at least 1:1 and no greater than 100:1 by mass.

In some embodiments, the composition comprises humulene epoxide I. In some specific embodiments, the composition comprises humulene and humulene epoxide I at a ratio of at least 1:1 and no greater than 10,000:1 by mass. In some very specific embodiments, the composition comprises humulene and humulene epoxide I at a ratio of at least 10:1 and no greater than 1,000:1 by mass.

In some embodiments, the composition comprises humulene and humulene epoxide II at a ratio of at least 1:10 and no greater than 1,000:1 by mass. In some specific embodiments, the composition comprises humulene and humulene epoxide II at a ratio of at least 1:1 and no greater than 100:1 by mass.

In some embodiments, the composition comprises humulene epoxide III. In some specific embodiments, the composition comprises humulene and humulene epoxide III at a ratio of at least 1:1 and no greater than 10,000:1 by mass. In some very specific embodiments, the composition comprises humulene and humulene epoxide III at a ratio of at least 10:1 and no greater than 1,000:1 by mass.

In some embodiments, the composition comprises bicyclohumuladiol. In some specific embodiments, the composition comprises humulene and bicyclohumuladiol at a ratio of at least 1:10 and no greater than 1,000:1 by mass. In some very specific embodiments, the composition comprises humulene and bicyclohumuladiol at a ratio of at least 1:1 and no greater than 100:1 by mass.

In some embodiments, the composition comprises tricyclohumuladiol. In some specific embodiments, the composition comprises humulene and tricyclohumuladiol at a ratio of at least 1:10 and no greater than 1,000:1 by mass. In some very specific embodiments, the composition comprises humulene and tricyclohumuladiol at a ratio of at least 1:1 and no greater than 100:1 by mass.

In some embodiments, the composition comprises tricyclohumuladiol II. In some specific embodiments, the composition comprises humulene and tricyclohumuladiol II at a ratio of at least 1:1 and no greater than 10,000:1 by mass. In some very specific embodiments, the composition comprises humulene and tricyclohumuladiol II at a ratio of at least 10:1 and no greater than 1,000:1 by mass.

In some embodiments, the composition comprises humulenol II. In some specific embodiments, the composition comprises humulene and humulenol II at a ratio of at least 1:1 and no greater than 10,000:1 by mass. In some very specific embodiments, the composition comprises humulene and humulenol II at a ratio of at least 10:1 and no greater than 1,000:1 by mass.

In some embodiments, the composition comprises cannabidiol, tetrahydrocannabinol, cannabichromene, cannabigerol, cannabinol, and cannabidivarin, wherein: the composition comprises the cannabidiol at a concentration of at least 1 percent and no greater than 99 percent by mass; the composition comprises the cannabidiol and the tetrahydrocannabinol at a ratio of at least 1:1 and no greater than 10,000:1 by mass; the composition comprises the cannabichromene and the tetrahydrocannabinol at a ratio of greater than 1:1 and no greater than 1,000:1 by mass; the composition comprises the cannabigerol and the tetrahydrocannabinol at a ratio of greater than 1:1 and no greater than 1,000:1 by mass; the composition comprises the cannabidiol and the cannabinol at a ratio of at least 100:1 and no greater than 10,000:1 by mass; and the composition comprises the cannabidiol and the cannabidivarin at a ratio of at least 10:1 and no greater than 100:1 by mass.

In some embodiments, the composition comprises cannabidiol at a concentration of greater than 50 percent by mass, wherein: the composition has a temperature; the cannabidiol has a freezing point; and the temperature of the composition is less than the freezing point of the cannabidiol. In some specific embodiments, the composition comprises cannabidiol at a concentration of greater than 50 percent and no greater than 75 percent by mass.

“Freezing point” refers to the greatest temperature at which either purified molecule (for example, cannabidiol) or a solution comprising two or more chemical species (for example, a composition) is thermodynamically stable as a solid, and “freezing point” is determined at atmospheric pressure.

In some embodiments, the composition has a freezing point; the composition lacks a glass transition temperature; and the composition is a supercooled liquid.

“Supercooled liquid” refers to a liquid that exists at a temperature that is lower than the freezing point of the liquid, for example, because the liquid is kinetically trapped in a liquid phase.

In some embodiments, the composition comprises crystals of either cannabigerovarin, cannabidivarin, cannabigerol, or cannabidiol. In some specific embodiments, the composition comprises crystals of cannabigerovarin. In some specific embodiments, the composition comprises crystals of cannabidivarin. In some specific embodiments, the composition comprises crystals of cannabigerol. In some specific embodiments, the composition comprises crystals of cannabidiol.

In some embodiments, the composition lacks a freezing point; the composition has a glass transition temperature; and the composition is a glass.

In some embodiments, the composition comprises a solvent selected from ethanol, propane, propane-2-ol, propane-1,2-diol, n-butane, n-pentane, hexanes (which consist of n-hexane optionally one or more structural isomers of n-hexane), n-heptane, and supercritical carbon dioxide. In some specific embodiments, the solvent is ethanol. In some specific embodiments, the solvent is propane. In some specific embodiments, the solvent is propane-2-ol. In some specific embodiments, the solvent is propane-1,2-diol. In some specific embodiments, the solvent is n-butane. In some specific embodiments, the solvent is n-pentane. In some specific embodiments, the solvent is hexanes. In some specific embodiments, the solvent is n-heptane. In some specific embodiments, the solvent is supercritical carbon dioxide.

In some embodiments, the composition comprises crystals of either cannabidiol, cannabidivarin, cannabigerol, or cannabigerovarin, wherein the beta-caryophyllene, caryophyllene oxide, humulene, and humulene epoxide II are solutes that are dissolved in the solvent.

In some embodiments, the composition comprises a solvent selected from a coconut oil, palm kernel oil, avocado oil, hempseed oil, sunflower seed oil, grapeseed oil, cottonseed oil, peanut oil, canola oil, olive oil, soybean oil, and a mixture of two or more of the preceding oils.

In some embodiments, the composition comprises the solvent at a concentration of greater than 50 percent by mass. In some specific embodiments, the composition comprises the solvent at a concentration of at least 60 percent and no greater than 98 percent by mass.

In some embodiments, the composition comprises cannabigerovarin at a concentration of at least 35 percent and no greater than 75 percent by mass. In some specific embodiments, the composition comprises cannabigerovarin at a concentration of at least 45 percent and no greater than 70 percent by mass.

In some embodiments, the composition comprises cannabidivarin at a concentration of at least 35 percent and no greater than 75 percent by mass. In some specific embodiments, the composition comprises cannabidivarin at a concentration of at least 45 percent and no greater than 70 percent by mass. In some specific embodiments, the composition further comprises both (6aR,10aR)-6,6,9-trimethyl-3-pentyl-6a,7,8,10a-tetrahydro-6H-benzo[c]chromen-1-ol and (6aS,10aR)-6,6,9-trimethyl-3-pentyl-6a,7,8,10a-tetrahydro-6H-benzo[c]chromen-1-ol.

In some embodiments, the composition comprises tetrahydrocannabivarin at a concentration of at least 35 percent and no greater than 75 percent by mass. In some specific embodiments, the composition comprises tetrahydrocannabivarin at a concentration of at least 45 percent and no greater than 70 percent by mass.

In some embodiments, the composition comprises cannabigerol at a concentration of at least 35 percent and no greater than 75 percent by mass. In some specific embodiments, the composition comprises cannabigerol at a concentration of at least 45 percent and no greater than 70 percent by mass.

In some embodiments, the composition comprises cannabidiol at a concentration of at least 35 percent and no greater than 75 percent by mass. In some specific embodiments, the composition comprises cannabidiol at a concentration of at least 45 percent and no greater than 70 percent by mass. In some specific embodiments, the composition further comprises (6aR,10aR)-6,6,9-trimethyl-3-propyl-6a,7,8,10a-tetrahydro-6H-benzo[c]chromen-1-ol and (6aS, 10aR)-6,6,9-trimethyl-3-propyl-6a,7,8,10a-tetrahydro-6H-benzo[c]chromen-1-ol.

In some embodiments, the composition comprises tetrahydrocannabinol at a concentration of at least 35 percent and no greater than 90 percent by mass. In some specific embodiments, the composition comprises tetrahydrocannabinol at a concentration of at least 45 percent and no greater than 85 percent by mass. In some very specific embodiments, the composition comprises tetrahydrocannabinol at a concentration of at least 50 percent and no greater than 80 percent by mass.

Various combinations of the features disclosed in this patent document will be evident to those of ordinary skill, and these combinations are expressly contemplated. This patent document discloses each linguistic and grammatical combination of features disclosed anywhere in the patent document as though any specific combination were disclosed in the same sentence except for combinations that lack internal consistency, which will be apparent to those of ordinary skill.

“Comprising” and “comprise(s)” refer to open-ended sets. A composition comprising cannabigerol, for example, can also comprise cannabidiol.

What is claimed is:

1. A composition, comprising cannabigerovarin, beta-caryophyllene, caryophyllene oxide, humulene, and humulene epoxide II, wherein:
the composition comprises the cannabigerovarin and the beta-caryophyllene at a ratio of at least 1:5 and no greater than 100:1 by mass;
the composition comprises the cannabigerovarin and the caryophyllene oxide at a ratio of at least 1:1 and no greater than 500:1 by mass;
the composition comprises the cannabigerovarin and the humulene at a ratio of at least 1:5 and no greater than 100:1 by mass; and
the composition comprises the cannabigerovarin and the humulene epoxide II at a ratio of at least 1:1 and no greater than 500:1 by mass.

2. A composition, comprising cannabidivarin, beta-caryophyllene, caryophyllene oxide, humulene, and humulene epoxide II, wherein:
the composition comprises the cannabidivarin and the beta-caryophyllene at a ratio of at least 1:5 and no greater than 100:1 by mass;
the composition comprises the cannabidivarin and the caryophyllene oxide at a ratio of at least 1:1 and no greater than 500:1 by mass;
the composition comprises the cannabidivarin and the humulene at a ratio of at least 1:5 and no greater than 100:1 by mass; and
the composition comprises the cannabidivarin and the humulene epoxide II at a ratio of at least 1:1 and no greater than 500:1 by mass.

3. A composition, comprising tetrahydrocannabivarin, beta-caryophyllene, caryophyllene oxide, humulene, and humulene epoxide II, wherein:
the composition comprises the tetrahydrocannabivarin and the beta-caryophyllene at a ratio of at least 1:5 and no greater than 100:1 by mass;
the composition comprises the tetrahydrocannabivarin and the caryophyllene oxide at a ratio of at least 1:1 and no greater than 500:1 by mass;
the composition comprises the tetrahydrocannabivarin and the humulene at a ratio of at least 1:5 and no greater than 100:1 by mass; and
the composition comprises the tetrahydrocannabivarin and the humulene epoxide II at a ratio of at least 1:1 and no greater than 500:1 by mass.

4. A composition, comprising cannabigerol, beta-caryophyllene, caryophyllene oxide, humulene, and humulene epoxide II, wherein:
the composition comprises the cannabigerol and the beta-caryophyllene at a ratio of at least 1:5 and no greater than 100:1 by mass;
the composition comprises the cannabigerol and the caryophyllene oxide at a ratio of at least 1:1 and no greater than 500:1 by mass;
the composition comprises the cannabigerol and the humulene at a ratio of at least 1:5 and no greater than 100:1 by mass; and
the composition comprises the cannabigerol and the humulene epoxide II at a ratio of at least 1:1 and no greater than 500:1 by mass.

5. A composition, comprising cannabidiol, beta-caryophyllene, caryophyllene oxide, humulene, and humulene epoxide II, wherein:
the composition comprises the cannabidiol and the beta-caryophyllene at a ratio of at least 1:1 and no greater than 500:1 by mass;
the composition comprises the cannabidiol and the caryophyllene oxide at a ratio of at least 10:1 and no greater than 5,000:1 by mass;
the composition comprises the cannabidiol and the humulene at a ratio of at least 1:1 and no greater than 500:1 by mass; and
the composition comprises the cannabidiol and the humulene epoxide II at a ratio of at least 10:1 and no greater than 5,000:1 by mass.

6. A composition, comprising tetrahydrocannabinol, beta-caryophyllene, caryophyllene oxide, humulene, and humulene epoxide II, wherein:
the composition comprises the tetrahydrocannabinol and the beta-caryophyllene at a ratio of at least 1:1 and no greater than 500:1 by mass;
the composition comprises the tetrahydrocannabinol and the caryophyllene oxide at a ratio of at least 10:1 and no greater than 5,000: 1 by mass;
the composition comprises the tetrahydrocannabinol and the humulene at a ratio of at least 1:1 and no greater than 500:1 by mass; and
the composition comprises the tetrahydrocannabinol and the humulene epoxide II at a ratio of at least 10:1 and no greater than 5,000: 1 by mass.

7. The composition of any one of claims 1-6, comprising cannabigerol, cannabidiol, tetrahydrocannabinol, and cannabichromene.

8. The composition of any one of claims 1-7, comprising cannabigerol and tetrahydrocannabinol at a ratio of greater than 1:1 and no greater than 1,000: 1 by mass.

9. The composition of any one of claims 1-8, comprising cannabidiol and cannabinol at a ratio of at least 100:1 and no greater than 10,000: 1 by mass.

10. The composition of any one of claims 1-9, comprising cannabigerovarin, cannabidivarin, tetrahydrocannabivarin, and cannabichromevarin.

11. The composition of any one of claims 1-10, comprising cannabigerovarin and tetrahydrocannabivarin at a ratio of greater than 1:1 and no greater than 1,000:1 by mass.

12. The composition of any one of claims 1-11, comprising cannabidivarin and cannabivarin at a ratio of at least 100:1 and no greater than 10,000: 1 by mass.

13. The composition of any one of claims 1-12, comprising beta-caryophyllene and caryophyllene oxide at a ratio of at least 1:1 and no greater than 1,000: 1 by mass.

14. The composition of any one of claims 1-13, comprising humulene epoxide I.

15. The composition of claim 14, comprising humulene and humulene epoxide I at a ratio of at least 1:1 and no greater than 10,000: 1 by mass.

16. The composition of any one of claims 1-15, comprising humulene and humulene epoxide II at a ratio of at least 1:10 and no greater than 1,000: 1 by mass.

17. The composition of any one of claims 1-16, comprising humulene epoxide III.

18. The composition of claim 17, comprising humulene and humulene epoxide III at a ratio of at least 1:1 and no greater than 10,000:1 by mass.

19. The composition of any one of claims 1-18, comprising bicyclohumuladiol.

20. The composition of claim 19, comprising humulene and bicyclohumuladiol at a ratio of at least 1:10 and no greater than 1,000: 1 by mass.

21. The composition of any one of claims 1-20, comprising tricyclohumuladiol.

22. The composition of claim 21, comprising humulene and tricyclohumuladiol at a ratio of at least 1:10 and no greater than 1,000: 1 by mass.

23. The composition of any one of claims 1-22, comprising tricyclohumuladiol II.

24. The composition of claim 23, comprising humulene and tricyclohumuladiol II at a ratio of at least 1:1 and no greater than 10,000:1 by mass.

25. The composition of any one of claims 1-24, comprising humulenol II.

26. The composition of claim 25, comprising humulene and humulenol II at a ratio of at least 1:1 and no greater than 10,000:1 by mass.

27. The composition of any one of claims 1-26, comprising cannabidiol, tetrahydrocannabinol, cannabichromene, cannabigerol, cannabinol, and cannabidivarin, wherein:
the composition comprises the cannabidiol at a concentration of at least 1 percent and no greater than 99 percent by mass;
the composition comprises the cannabidiol and the tetrahydrocannabinol at a ratio of at least 1:1 and no greater than 10,000: 1 by mass;
the composition comprises the cannabichromene and the tetrahydrocannabinol at a ratio of greater than 1:1 and no greater than 1,000: 1 by mass;
the composition comprises the cannabigerol and the tetrahydrocannabinol at a ratio of greater than 1:1 and no greater than 1,000: 1 by mass;
the composition comprises the cannabidiol and the cannabinol at a ratio of at least 100:1 and no greater than 10,000:1 by mass; and
the composition comprises the cannabidiol and the cannabidivarin at a ratio of at least 10:1 and no greater than 100:1 by mass.

28. The composition of any one of claims 1-27, comprising cannabidiol at a concentration of greater than 50 percent by mass, wherein:
the composition has a temperature;
the cannabidiol has a freezing point; and
the temperature of the composition is less than the freezing point of the cannabidiol.

29. The composition of any one of claims 1-28, wherein the composition has a freezing point; the composition lacks a glass transition temperature; and the composition is a supercooled liquid.

30. The composition of any one of claims 1-28, comprising crystals of cannabidiol.

31. The composition of any one of claims 1-27, wherein the composition lacks a freezing point; the composition has a glass transition temperature; and the composition is a glass.

32. The composition of any one of claims 1-26, comprising crystals of cannabidivarin, cannabigerol, or cannabigerovarin.

33. The composition of any one of claims 1-32, comprising a solvent selected from ethanol, propane, propane-2-ol, propane-1,2-diol, n-butane, n-pentane, hexanes, n-heptane, and supercritical carbon dioxide.

34. The composition of claim 33, comprising crystals of either cannabidiol, cannabidivarin, cannabigerol, or cannabigerovarin, wherein the beta-caryophyllene, caryophyllene oxide, humulene, and humulene epoxide II are solutes that are dissolved in the solvent.

35. The composition of any one of claims 1-32, comprising a solvent selected from a coconut oil, palm kernel oil, avocado oil, hempseed oil, sunflower seed oil, grapeseed oil, cottonseed oil, peanut oil, canola oil, olive oil, soybean oil, and a mixture of two or more of the preceding oils.

36. The composition of any one of claims 33-35, comprising the solvent at a concentration of greater than 50 percent by mass.

37. The composition of any one of claims 1-34, comprising cannabigerovarin at a concentration of at least 35 percent and no greater than 75 percent by mass.

38. The composition of any one of claims 1-34, comprising cannabidivarin at a concentration of at least 35 percent and no greater than 75 percent by mass.

39. The composition of claim 38, comprising (6aR,10aR)-6,6,9-trimethyl-3-pentyl-6a,7,8,10a-tetrahydro-6H-benzo[c]chromen-1-ol and (6aS,10aR)-6,6,9-trimethyl-3-pentyl-6a,7,8,10a-tetrahydro-6H-benzo[c]chromen-1-ol.

40. The composition of any one of claims 1-33, comprising tetrahydrocannabivarin at a concentration of at least 35 percent and no greater than 75 percent by mass.

41. The composition of any one of claims 1-34, comprising cannabigerol at a concentration of at least 35 percent and no greater than 75 percent by mass.

42. The composition of any one of claims 1-34, comprising cannabidiol at a concentration of at least 35 percent and no greater than 75 percent by mass.

43. The composition of claim 42, comprising (6aR,10aR)-6,6,9-trimethyl-3-propyl-6a,7,8,10a-tetrahydro-6H-benzo[c]chromen-1-ol and (6aS,10aR)-6,6,9-trimethyl-3-propyl-6a,7,8,10a-tetrahydro-6H-benzo[c]chromen-1-ol.

44. The composition of any one of claims 1-33, comprising tetrahydrocannabinol at a concentration of at least 45 percent and no greater than 85 percent by mass.
